(19) United States
(12) Patent Application Publication
(10) Pub. No.: US 2025/0342401 A1
(43) Pub. Date: Nov. 6, 2025

(54) LEARNING CELLULAR AUTOMATA

(71) Applicant: University of Florida Research Foundation, Incorporated, Gainesville, FL (US)

(72) Inventors: Jose C. Principe, Gainesville, FL (US); Naoki Sawahashi, Gainesville, FL (US)

(21) Appl. No.: 19/196,243

(22) Filed: May 1, 2025

Related U.S. Application Data
(60) Provisional application No. 63/641,559, filed on May 2, 2024.

(51) Int. Cl. G06N 20/00; G06V 10/764; G16B 40/00

(52) U.S. Cl. CPC G06N 20/00; G06V 10/764; G16B 40/00

(57) ABSTRACT

Various embodiments of the present disclosure provide for learning cellular automata. In one example, an embodiment provides for determining a lattice data structure that defines a plurality of cells associated with cellular automata. In another example, an embodiment provides for partitioning the lattice data structure into (i) an input region associated with input data, (ii) an output region associated with output data, and (iii) a processing region associated with a cost function for the cellular automata. In another example, an embodiment provides for training an adaptive lattice model associated with cellular automata based on the partitioned lattice data structure.

### CROSS REFERENCE TO RELATED APPLICATIONS

This application claims priority to U.S. Appl. No. 63/641,559 filed May 2, 2024, the contents of which are incorporated herein in its entirety by reference.

### GOVERNMENT SUPPORT

This invention was made with government support under N00014-21-1-2324 awarded by the US NAVY OFFICE of NAVAL RESEARCH. The government has certain rights in the invention.

### TECHNICAL FIELD

The present application relates to the technical field of artificial intelligence and/or image classification. In particular, the invention relates to computation intelligence for learning cellular automata.

### BACKGROUND

Cellular automaton is a model that performs one or more computations over a cellular lattice. However, it is desirable to improve utilization of cellular automata for real-world computing applications.

### SUMMARY

In general, embodiments of the present invention provide methods, apparatus, systems, computing devices, computing entities, and/or the like that provide for learning cellular automata. The details of some embodiments of the subject matter described in this specification are set forth in the accompanying drawings and the description below. Other features, aspects, and advantages of the subject matter will become apparent from the description, the drawings, and the claims.

In an embodiment, a method for providing machine learning associated with cellular automata is provided. The method provides for determining a lattice data structure that defines a plurality of cells associated with cellular automata. The method additionally or alternatively provides for partitioning the lattice data structure into (i) an input region associated with input data, (ii) an output region associated with output data, and (iii) a processing region associated with a cost function for the cellular automata. The method additionally or alternatively provides for training an adaptive lattice model associated with cellular automata based on the partitioned lattice data structure.

In another embodiment, an apparatus for providing a functional verification flow of obfuscated designs for circuits is provided. The apparatus comprises at least one processor and at least one memory including program code. The at least one memory and the program code are configured to, with the at least one processor, cause the apparatus to determine a lattice data structure that defines a plurality of cells associated with cellular automata. The at least one memory and the program code are additionally or alternatively configured to, with the at least one processor, further cause the apparatus to partition the lattice data structure into (i) an input region associated with input data, (ii) an output region associated with output data, and (iii) a processing region associated with a cost function for the cellular automata. The at least one memory and the program code are additionally or alternatively configured to, with the at least one processor, further cause the apparatus to train an adaptive lattice model associated with cellular automata based on the partitioned lattice data structure.

In yet another embodiment, a non-transitory computer storage medium comprising instructions for providing a functional verification flow of obfuscated designs for circuits is provided. The instructions are configured to cause one or more processors to perform operations configured to determine a lattice data structure that defines a plurality of cells associated with cellular automata. The instructions are additionally or alternatively configured to cause one or more processors to perform operations configured to partition the lattice data structure into (i) an input region associated with input data, (ii) an output region associated with output data, and (iii) a processing region associated with a cost function for the cellular automata. The instructions are additionally or alternatively configured to cause one or more processors to perform operations configured to train an adaptive lattice model associated with cellular automata based on the partitioned lattice data structure.

### BRIEF DESCRIPTION OF THE DRAWINGS

Reference will now be made to the accompanying drawings, which are not necessarily drawn to scale, and wherein:

FIG. 1 provides an example framework for genetic encoding for linear hybrid cellular automata, according to one or more embodiments of the present disclosure;

FIG. 2 illustrates an example flow diagram related to learning cellular automata of a genetic algorithm with elitism, according to one or more embodiments of the present disclosure;

FIG. 3 illustrates an example system of different lattice topologies specified by placement of motor cells, according to one or more embodiments of the present disclosure;

FIG. 4 illustrates an example dataflow for training an adaptive lattice model, according to one or more embodiments of the present disclosure;

FIG. 5A illustrates a flowchart of a method for providing machine learning associated with cellular automata according to one or more embodiments of the present disclosure;

FIG. 5B illustrates a flowchart of a method for training an adaptive lattice model associated with cellular automata according to one or more embodiments of the present disclosure;

FIG. 6 illustrates an exemplary overview of an architecture that can be used to practice one or more embodiments of the present disclosure;

FIG. 7 illustrates a schematic of a computing entity that may be used in conjunction with one or more embodiments of the present disclosure;

FIG. 8 illustrates an example client computing entity in accordance with one or more embodiments of the present disclosure;

FIG. 9 illustrates example topologies for a partitioned lattice data structure in accordance with one or more embodiments of the present disclosure; and

FIG. 10 illustrates average mutual information between each cell and the fitness value for an operator in accordance with one or more embodiments of the present disclosure.

### DETAILED DESCRIPTION

The present disclosure more fully describes various embodiments with reference to the accompanying drawings. It should be understood that some, but not all, embodiments are shown and described herein. Indeed, the embodiments may take many different forms, and, accordingly, this disclosure should not be construed as limited to the embodiments set forth herein. Rather, these embodiments are provided so that this disclosure will satisfy applicable legal requirements. Like numbers refer to like elements throughout.

Cellular automaton is a model (e.g., an autonomous dynamical system) that is discrete in time, space, and state space. Typically, cellular automaton can perform one or more computations over a cellular lattice. For example, cellular automata can utilize processing techniques inspired by anatomical properties of the cortical column such as, for example, excitatory (e.g., pyramidal cells) and inhibitory neurons, to provide scalable and efficient computing related to parallelism of the cortex. Cellular automata typically consist of multiple agents (e.g., cells) and is typically defined over a one-dimensional (1D) or two-dimensional (2D) Cartesian lattice. At each time step, respective cells update a respective state based on a pre-defined rule. Additionally respective cells may receive a cell previous state and/or a state of neighbor cells as input. If all cells follow the same state transition rule and share a single neighborhood pattern, the cellular automata may be homogeneous. Additionally, certain uniform cellular automata may be chaotic and develop complex self-similar structures over space and time, referred to as Class 4 cellular automata. With Class 4 cellular automata, universality is achieved by coding a machine (e.g., a glider) and data in an initial state. As such, Class 4 uniform cellular automata is typically inefficient both in space (e.g., the lattice size) and time (e.g., clock cycles) as a medium for an associative memory. Additionally, a lack of and/or inefficient training techniques for cellular automata (e.g., elementary cellular automata, reversible cellular automata, hexagonal cellular automata, triangular cellular automata, hybrid cellular automata, multi-attractor cellular automata, cellular automata network, etc.) typically minimizes utilization of cellular automata for real-world computing applications. For example, typical cellular automata models lack training techniques for computing a rule vector that evolves initial states/inputs to desired attractors. Additionally, building a state transition diagram for a cellular automaton is typically not scalable to a large lattice since only recursive numerical simulations may determine a trajectory of an arbitrary cellular automata.

To address these and/or other issues, various embodiments described herein relate to learning cellular automata. Various embodiments described herein may enable cellular automata to extend to machine learning applications. For example, various embodiments described herein may provide machine learning associated with cellular automata. The machine learning associated with cellular automata may enable scalable and/or efficient computing related to machine learning. In various embodiments, end-to-end image classification in linear hybrid cellular automata is provided. In various embodiments, a cellular lattice can be partitioned into an input region, an output region, and/or a processing region. Additionally, a synthesis technique can be utilized to train a linear hybrid cellular automaton for arbitrary input-output maps. Based on the trained cellular automaton, image classification with respect to a dataset can be provided. For example, a trained cellular automaton may enable utilization of cellular automata for image classification for a dataset. In some embodiments, a synthesis framework can be provided based on the trained cellular automaton for performing end-to-end classification in a dataset. In various embodiments, by mapping local states over a globally linear lattice, the trained cellular automaton can provide improved accuracy for binary image classification. In various embodiments, operation of the trained cellular automaton can be defined by a look-up table (LUT) rather than a combination of arithmetic operators (e.g., multiplication). Additionally, the trained cellular automaton can be executed without utilization of pre-processors and/or post-processors to perform computations over the lattice. For example, the trained cellular automaton can be executed without an external post-processor to retrieve a label from the lattice. Hence, a lattice can maintain massive parallelism and locality of computation for ultra-low power processing related to machine learning.

In various embodiments, a trained cellular automaton can be utilized as an ultra-low power, alternative processing element for machine learning. For example, a trained cellular automaton can be utilized as a processor for energy-efficient machine learning. Accordingly, improved image classification, parallel processing, neuromorphic computing, and/or machine learning can be provided. For example, the trained cellular automaton can be executed without experiencing a von-Neumann bottleneck. Additionally, the trained cellular automaton can be operated in parallel (e.g., the cellular automaton can be run in parallel) due to respective cells being autonomous and/or modular. In various embodiments, the trained cellular automaton may enable utilization of cellular automata for real-world computing applications. For example, the trained cellular automaton may enable utilization of cellular automata for a machine learning task. In various embodiments, the trained cellular automaton provides reduced computing time and/or reduced power consumption as compared to traditional processors and/or traditional machine learning models.

a. Example Cellular Automata of Various Embodiments

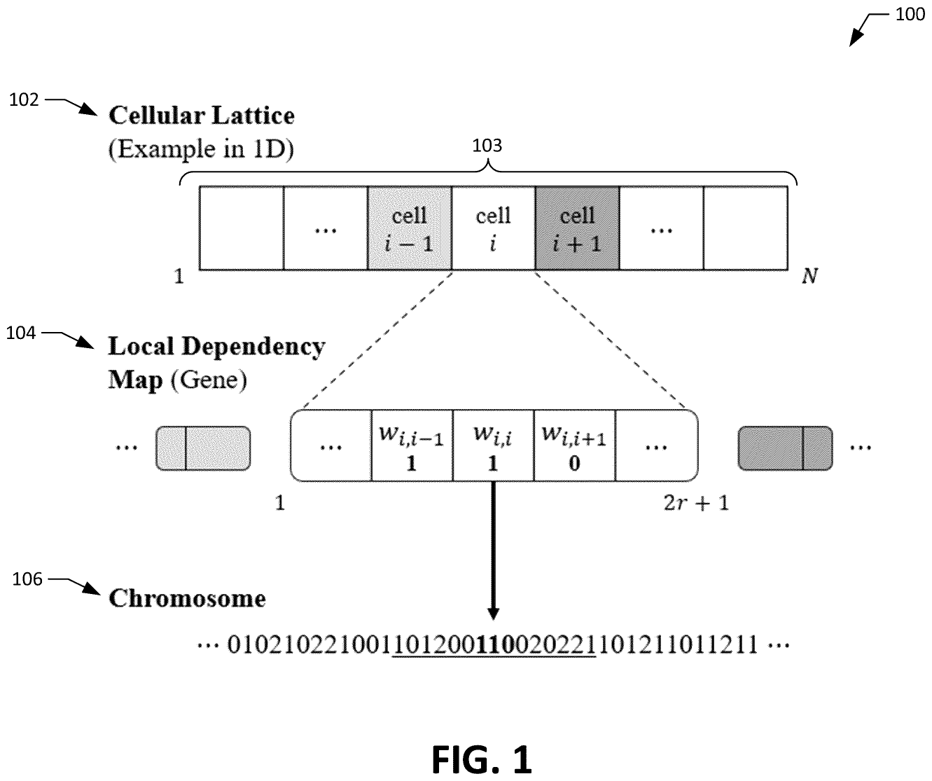

A framework 100 for genetic encoding for linear hybrid cellular automata is shown in FIG. 1, according to one or more embodiments of the present disclosure. In various embodiments, the framework 100 can provide an encoding scheme for a cellular automaton as a chromosome through local dependency maps. In some examples, the framework 100 can be a learning framework to achieve end-to-end image classification in linear hybrid cellular automata. In various embodiments, the framework 100 can operate on a look-up table of a cell to provide parallelism and computational locality for machine learning. For example, cellular automata can be implemented based on a look-up table (e.g., logic gates). Additionally, computation can be accomplished based on dynamic interactions between cells in the lattice. For example, conditions of a local dynamical equation stored in respective cells can be dynamically updated based on dynamic interactions between cells in the lattice. In various embodiments, cellular automata can be configured to interact with nearest neighbors, thereby avoiding computational inefficiencies such as, for example, Von Neumann bottlenecks. In various embodiments, the framework 100 can provide an adaptive lattice model and/or cellular automata that can learn arbitrary input-output maps from data, related to machine learning.

In various embodiments, a cellular automaton can be trained to match arbitrary input output maps. In various embodiments where a characteristics matrix is non-singular, an additive cellular automaton that guarantees that the dynamics avoid a zero global attractor can be utilized, while limiting the exponential growth of the parameter space. For example, the exponential growth of a dynamic range (e.g., the size of the state set) and/or local connectivity (e.g., neighborhood radius) can be minimized.

A non-uniform cellular automaton can be, for example, a hybrid cellular automaton where cells are at least approximately homogeneous. In certain embodiments, a hybrid cellular automaton can comprise different transition rules. In various embodiments, a hybrid cellular automaton can be defined by the following tuple:

$$\{ \begin{matrix}Σ & a set of states \\ {η}_{i} & neighbors \\ {ϕ}_{i} : {Σ}^{\vert {η}_{i} \vert} \to Σ & a look - up table ( LUT ) \\ S ( 0 ) & an initial state of a lattice\end{matrix} \qquad ( 1 )$$

In various embodiments, a 1D lattice over a Galois Field (GF) of an order k can be utilized. Additionally, a synchronous update can be employed such that the states of all cells are updated simultaneously. The ith cell's neighbor can be represented by the following:

$${η}_{i} = [ {s}_{i - r} , … , {s}_{i - 1} , {s}_{i} , {s}_{i + 1} , … , {s}_{i + r} ] \qquad ( 2 )$$

where r is a radius, and i is a location of the cell in the lattice, indexed from 1 to N where N corresponds to a number of cells that a cellular automaton comprises (e.g., a size of the lattice). In various embodiments, a Wolfram's elementary cellular automata notation can be utilized to express a LUT in which a bit/ternary specifies an output for each input combination. Additionally, a bit string (e.g., of length k^{k2r+1}) can be converted to a positive integer in base 10.

In various embodiments, additivity in a global state transition of a linear hybrid cellular automaton can be preserved as shown by the following:

$${∀}_{t > 0} , {ϕ}^{t} \{ {S}_{A} ( 0 ) + {S}_{B} ( 0 ) \} = {ϕ}^{t} \{ {S}_{A} ( 0 ) \} + {ϕ}^{t} \{ {S}_{B} ( 0 ) \} \qquad ( 3 )$$

In various embodiments, a state space Σ^{N} can be defined over any finite set 2 such that a ring is formed to define the additivity in the global state transition. One such set is a GF, where the standard addition and multiplication operators are used over mod (k). Addition is defined as XOR operator for k=2. For r=1, there can be 8 XOR rules over GF and 8 of their complements, as summarized below in Table I given the following:

$${s}_{i} ( t ) = {w}_{i - 1} {s}_{i - 1} ( t - 1 ) + {w}_{i} {s}_{i} ( t - 1 ) + {w}_{i + 1} {s}_{i + 1} ( t - 1 ) \qquad ( 4 )$$

TABLE I
XOR/XNOR RULES FOR r = 1

| | Rule Number | | | | | | |
|---|---|---|---|---|---|---|---|
| | 60 | 90 | 102 | 150 | 170 | 204 | 240 |
| | (Complements) | | | | | | |
| | (195) | (165) | (153) | (105) | (85) | (51) | (15) |
| wᵢ₋₁ | 1 | 1 | 0 | 1 | 0 | 0 | 1 |
| wᵢ | 1 | 0 | 1 | 1 | 0 | 1 | 0 |
| wᵢ₊₁ | 0 | 1 | 1 | 1 | 1 | 0 | 0 |

*Rule 0 (255) is omitted.

In various embodiments, these rules can be expressed by bits specifying the dependency among neighbors. For any k, a N×N characteristic matrix can be defined as:

$$T = [ \begin{matrix}{w}_{1 , 1} & {w}_{1 , 2} & 0 & … & … & 0 \\ {w}_{1 , 2} & {w}_{2 , 2} & {w}_{2 , 3} & 0 & … & 0 \\ ⋮ & ⋮ & ⋮ & ⋮ & ⋱ & 0 \\ 0 & … & … & 0 & {w}_{N , N - 1} & {w}_{N , N}\end{matrix} ] \qquad ( 5 )$$

Based on equations (3), (4), and (5), the global state transition can be represented by the following:

$$S ( t ) = T \cdot S ( t - 1 ) \qquad ( 6 )$$

$$S ( t + 1 ) = T \cdot T \cdot S ( t )$$

$$⋮$$

$$S ( t + τ ) = {T}^{τ} \cdot S ( 0 )$$

where S(t) is a N× 1 state vector of the lattice at t. It is to be appreciated that equation (6) shows various advantages of the linear hybrid cellular automata such as, for example, that a global state of the lattice can be computed without recursive computations. Accordingly, a number of computing resources for deciphering the relationship between a rule vector and the dynamics it generates can be reduced. By utilizing equation (6), a global state X is a (point) attractor if:

$$( T + I ) \cdot X = 0 \qquad ( 7 )$$

Accordingly, equations (5) to (7) illustrate advantages of linear cellular automata such as, for example, that the rank of the matrix T+I determines the number of attractors, Tis invertible if and only if all trajectories are on limit cycles, the factor(s) of a characteristic polynomial T+xI determines a length of the cycle(s), and an ith ternary of an attractor defines possible rules for the ith cell. In various embodiments, a rule vector can belong to Cartesian products of such rule sets to facilitate partitioning of a lattice.

In various embodiments, the cell population can be partitioned into receptors (e.g., inputs), motors (e.g., outputs), and/or processors. In various embodiments, a local mapping can be defined from receptors to motors over space and time rather than a mapping (e.g., a global mapping) between lattice states over time that is limited to a linear transformation. In various embodiments, receptors and motors can be utilized for write-in and read-out of the system. In various embodiments, processors can propagate and/or modulate activation provided by neighbor cells (e.g., neighbor processors).

In a non-limiting example, an Mₓ-dimensional input sample x and M_{y}-dimensional target d can be provided. Additionally, ξ can represent a Mₓ-dimensional vector specifying a location of the receptors over the lattice. An M_{y}-dimensional vector can be represented by o and can define a location of the motors. Given xⱼ=S_{ξ}(0) and yⱼ=Sₒ(t), the goal of synthesis can be to determine three vectors: ϕ, ξ and o, minimizing a cost function:

$$D = {\sum }_{j} \vert {ϕ}_{o}^{τ} \{ {S}_{ξ} ( 0 ) \} - {d}_{j} \vert \qquad ( 8 )$$

where j is an index of the training samples.

As shown in equation (8), an input space can be decoupled from a state space. Therefore, a lattice size N can be any number bigger than or equal to max (Mₓ, M_{y}). Accordingly, improved flexibility can be provided to allow a number of processing elements to be selected based on complexity of the mappings. For example, a smaller lattice can be desirable in hardware to reduce a number of transistors and computational time. Given the same neighborhood, the activation takes more time to propagate over every cell in a larger lattice. Meanwhile, increasing the neighborhood expands a LUT.

In various embodiments, a look-up table can be optimized for desired input-output maps via synthesis. For example, in various embodiments, the framework 100 may utilize a genetic algorithm with elitism to synthesize cellular automata. Additionally or alternatively, a size of a lattice (e.g., the lattice size N) can be adjusted via synthesis. In various embodiments, every cell of a linear hybrid cell automaton may utilize 2r+1 values to define an operation of the cellular automaton. As defined by equation (5), a 2r+1 vector of wᵢ (e.g., a local dependency map (LDM)) can be utilized to define the LUT operation.

The framework 100 illustrated in FIG. 1 includes a lattice 102. The lattice 102 can be a cellular lattice. In some examples, the lattice 102 can be a 1D Cartesian lattice over a GF. However, in certain embodiments, the lattice 102 can be a 2D Cartesian lattice. In various embodiments, the lattice 102 can be associated with a plurality of cells 103 (e.g., cell i−1, cell i, cell i+1, etc.).

In various embodiments, the lattice 102 can be partitioned into an input (e.g., sensory) region that receives inputs from one or more external sources, an output (e.g., motor) region that provides output using a winner-take-all operator, and a computational region (e.g., a processor) that maps the input region and the output region. As such, cellular based computation can be utilized while also providing the ability to fine-tune the lattice 102 for a specific task, thereby reducing a number of transistors and computing time for cellular automata. In various embodiments, in order to train a look-up table, a genetic algorithm (e.g., a training process) that finds a local structure of the look-up table (e.g., a string of ternary logic elements) can be utilized.

Additionally, the framework 100 includes an LDM 104 to define a LUT operation for a cellular automaton (e.g., a linear hybrid cellular automaton). For example, the LDM 104 can be associated with a particular cell (e.g., cell i) of the plurality of cells 103. In various embodiments, the LDM 104 can be encoded as a gene. For example, a chromosome 106 can consist of N genes with 2r+1 nucleotides. Each nucleotide can utilize a three-valued logic with a value of 0, 1, or 2, where 0 codes an inhibitory connection, 1 is a weak coupling, and 2 is a strong connection.

In various embodiment, the framework 100 can utilize a cost function for the cellular automaton. For example, a winner-take-all operator can be utilized to implement equation (8) for classification. In a non-limiting example, assume M samples consisting of K classes. Let j index a training sample from 1 to M, and i address the motor cells from 1 to K. As such, each chromosome can be realized as a cellular automaton (e.g., a linear hybrid cellular automaton). Additionally, each chromosome can be evolved to t time steps. The winner-take-all operation can then be applied to the states of the motor component at t. For example, let iₘₐₓ be an index of the motor whose value is the highest among the motor partition. As such, if iₘₐₓ is unique and matches with a label dj, the chromosome can be considered fit for sample j. In various embodiments, the overall fitness of a training batch is defined as:

$$F = \frac{1}{M} {\sum }_{j}^{M} {f}_{j} \qquad ( 9 )$$

$${f}_{j} = \{ \begin{matrix}1 & if {i}_{max} = {d}_{j} and {∀}_{i \neq imax} , i \neq {i}_{max} \\ 0 & otherwise\end{matrix}$$

It is to be appreciated that all fitness values can be evaluated during synthesis. In various embodiments, F can be equivalent to classification accuracy.

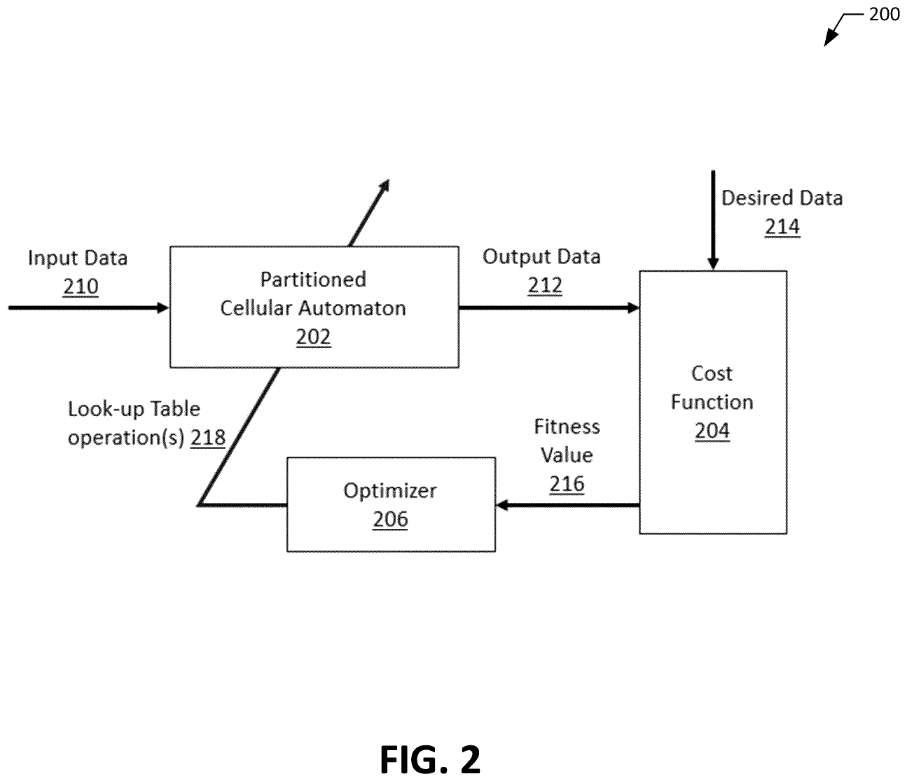

A system 200 related to learning cellular automata is shown in FIG. 2, according to one or more embodiments of the present disclosure. In various embodiments, the system 200 can provide training of an adaptive lattice model via learning cellular automata. For example, an adaptive lattice model associated with cellular automata can be trained based on a partitioned lattice data structure. In various embodiments, a training process associated with the system 200 can be data-driven (e.g., via supervised learning) to enable a cellular automaton to be trained for a machine learning task such as image classification or another type of machine learning task. The system 200 includes a partitioned cellular automaton 202 and/or an optimizer 206. In various embodiments, the optimizer 206 can optimize the partitioned cellular automaton 202 based on a cost function 204.

In various embodiments, the partitioned cellular automaton 202 can be an adaptive lattice model that is trained based on a partitioned lattice data structure. For example, the partitioned cellular automaton 202 can be a computational model associated with cellular automata. In some embodiments, the partitioned cellular automaton 202 can be a trained cellular automata for a computing task where the trained cellular automata includes multiple processing elements (e.g., cells) over a lattice space. Each processing element (e.g., each cell) can receive its neighbors' state as an input. Additionally, each processing element (e.g., each cell) can evolve its state based on a defined LUT. In some embodiments, the partitioned cellular automaton 202 can be trained such that local states are mapped over a globally linear lattice associated with the partitioned lattice data structure. In some embodiments, the partitioned cellular automaton 202 can be configured with tuned parameters associated with cortical column functionality that is free from von-Neumann bottlenecks. In some embodiments, operation of the partitioned cellular automaton 202 can be defined by a LUT that is tuned according to one or more look-up tables operations. In some embodiments, the optimizer 206 can update and/or tune look-up table operation(s) 218 of one or more LUTs for the partitioned cellular automaton 202. In some embodiments, the optimizer 206 can utilize a random search technique to update and/or tune look-up table operation(s) 218 of one or more LUTs for the partitioned cellular automaton 202. In some embodiments, the optimizer 206 can utilize a genetic algorithm to update and/or tune look-up table operation(s) 218 of one or more LUTs for the partitioned cellular automaton 202. In some embodiments, the optimizer 206 can utilize a random search technique to update and/or tune look-up table operation(s) 218 of one or more LUTs for the partitioned cellular automaton 202. In some embodiments, the optimizer 206 can utilize simulated annealing to update and/or tune look-up table operation(s) 218 of one or more LUTs for the partitioned cellular automaton 202. However, it is to be appreciated that, in some embodiments, the optimizer 206 can utilize a different type of optimization technique to update and/or tune look-up table operation(s) 218 of one or more LUTs for the partitioned cellular automaton 202.

In some embodiments, the partitioned cellular automaton 202 can encode a LUT of each cell as a local dependency map. Additionally, the partitioned cellular automaton 202 can embed input data 210 as initial states of receptor cells for the partitioned cellular automaton 202. In various embodiments, the partitioned cellular automaton 202 can be evolved and/or a winner-take-all function can be applied on the states of the motor cells to determine output data 212 of the partitioned cellular automaton 202. The output data 212 and desired data 214 can be utilized as input to the cost function 204 to provide a fitness value 216. The fitness value 216 provided by the cost function 204 can be utilized by the optimizer 206 to update and/or tune look-up table operation(s) 218 of one or more LUTs for the partitioned cellular automaton 202. For example, the optimizer 206 can update local dependency maps for the partitioned cellular automaton 202 based on the fitness value 216 provided by the cost function 204.

In various embodiments, the partitioned cellular automaton 202 can be trained for learning cellular automata based on one or more optimizers and/or search algorithms. For example, the one or more optimizers and/or search algorithms can be applicable to a particular model related to learning cellular automata and/or lattice partitioning. In various embodiments, genetic encoding for linear hybrid cellular automata and/or an application to the cost function 204 (e.g., cost function D) can be utilized to implement learning cellular automata. In various embodiments, tunable parameters of genetic algorithm operators can be specified and/or utilized for training associated with learning cellular automata. In a non-limiting example, hyperparameters can be configured such that P=100, p_{E}=0.1, p_{Xross}=0.45, and p_{Mut}=0.05, where P corresponds to population size, p_{E} corresponds to an elitism parameter, p_{Xross} corresponds to a crossover probability, and p_{Mut} corresponds to a mutation probability.

In various embodiments, chromosomes realized as a cellular automaton (e.g., a linear hybrid cellular automaton) can be initially ranked based on fitness. The chromosomes can include a population size P. In various embodiments, the top p_{E} portion of the fittest chromosomes can be copied to a next-generation pool. Additionally, the rank-based roulette-wheel selection of equation [8] can be applied to the entire current population P. A probability of a chromosome i being selected is given by:

$${p}_{i} = \frac{P - Rank ( i ) + 1}{{\sum }^{Q} Rank ( j )} \qquad ( 10 )$$

In various embodiments, the selected chromosomes can be copied to an intermediate population and randomly marked for a crossover given p_{Xover}. Additionally, marked chromosomes can be randomly paired and applied to a two-point crossover. In various embodiments, all nucleotides in the intermediate population can be mutated with a probability of p_{Mut}. A mutation can be performed by replacing a selected nucleotide with a random value drawn from a uniform distribution of [0, 2]∈Z. In various embodiments, the intermediate population can be moved to the remainder of the next generation. As such, tunable parameters (e.g., hyperparameters) may be defined for an operation of a cellular automaton. For example, tunable parameters (e.g., hyperparameters) may be trainable parameters and/or weights to synthesize a cellular automaton. Synthesis of a cellular automaton can include configuration of the cellular automaton to provide desirable functionality for a computing task. In some embodiments, a computing task can include a machine learning task. In some embodiments, a computing task can include an image classification task. In some embodiments, the training for the partitioned cellular automaton 202 can be repeated until the fitness (e.g. the fitness value 216) satisfies an early termination condition or exceeds a generation limit. Accordingly, genetic encoding of a linear hybrid cellular automata with a winner-take-all cost function can be provided. Additionally, a learning scheme to adapt nonlinear local mapping over a globally linear lattice can also be provided.

In some embodiments, synthesis of cellular automaton can include identifying one or more LUTs that optimally mimic an input-output map associated with data points. For example, synthesis of cellular automaton can involve identification of a set of LUTs that map receptor states to motor states, thereby differentiating input classes.

In an example where input corresponds to M image label pairs, synthesis may maximize the fitness function:

$$F = \frac{1}{M} {\sum }_{j}^{M} {f}_{j}$$

$${f}_{j} = \{ \begin{matrix}1 & if {y}_{j} = {d}_{j} and {y}_{j} \neq 0 \\ 0 & otherwise\end{matrix}$$

Thus, the locations of the motor cells and receptors may be associated with tunable parameters.

In some embodiments, a genetic algorithm (GA) associated with training of the partitioned cellular automaton 202 associated with an adaptive lattice model based on the following:

| GA | LHCA | Quantity |
|---|---|---|
| Nucleotide | Weight (w_{i, j}) | \| [symbol] \| |
| Gene | LUT (ϕ [symbol] ) | N |
| Chromosome | Lattice | P |

[symbol]

indicates data missing or illegible when filed

In some embodiments, P chromosomes can be randomly generated as an initial population. Each chromosome can be realized as a partitioned lattice data structure and evaluated on training data. In come embodiments, the chromosomes can be ranked by their fitness values, and the top p_{Elite} portion of the population can be copied to the next population without any modification. In some embodiments, a rank-based selection can be applied to the population to form an intermediate population. Within the intermediate population, the chromosomes can be randomly selected for crossover and mutation, specified by the preset probabilities p_{Xross} and p_{Mut}, respectively. In some embodiments, the intermediate population can be moved to the remainder of the next population. The evolution process can continue until a preset maximum generation Q is reached or the best fitness within the population becomes 1.

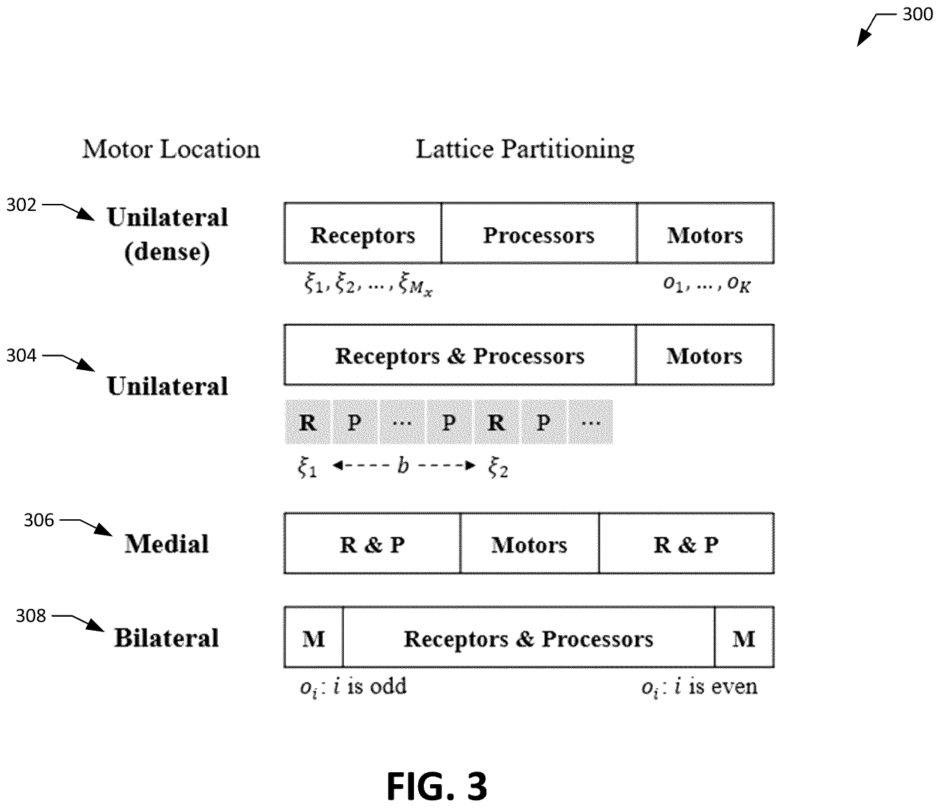

A system 300 of different lattice topologies specified by placement of motor cells is shown in FIG. 3, according to one or more embodiments of the present disclosure. For example, a lattice topology (e.g., a topology of the lattice 102) can be optimized based on spatial arrangement of a lattice. In this regard, the system 300 includes a topology 302 associated with a unilateral (dense) motor location, a topology 304 associated with a unilateral motor location, a topology 306 associated with a medial motor location, and a topology 308 associated with a bilateral motor location. In a non-limiting embodiment, spacing between receptors and processors can be set to 3 or another spacing value.

Receptors may correspond to an input region of a lattice data structure, motors may correspond to an output region of the lattice data structure, and processors may correspond to a processing region. For example, an input region can be an area or subset of cells configured to receive input data, an output region can be an area or subset of cells configured to provide output data, and a processing region can be an area or subset of cells configured to provide processing, computation, and/or state transitions.

In some embodiments, the topology 302 of a lattice data structure can include a lattice partitioning where receptors (e.g., an input region) are located proximate to processors (e.g., a processing region. Additionally, the processors (e.g., processing region) can be located proximate to motors (e.g., an output region). As such, the processors (e.g., processing region) can separate the receptors (e.g., input region) and motors (e.g., output region) in the topology 302.

In some embodiments, the topology 304 of a lattice data structure can include a lattice partitioning where receptors (e.g., an input region) and processors (e.g., a processing region) are included in a corresponding region where respective receptors are separated by respective processors. Additionally, the corresponding region associated with the receptors and processors can be located proximate to motors (e.g., an output region).

In some embodiments, the topology 306 of a lattice data structure can include a lattice partitioning with a first region, a second region, and a third region. The first region can include receptors (e.g., an input region) and processors (e.g., a processing region). Additionally, the third region can include receptors (e.g., an input region) and processors (e.g., a processing region). The second region can correspond to motors (e.g., output region). In some embodiments, respective receptors are separated by respective processors in the first region and/or the third region. The first region can be located proximate to the second region. Additionally, the second region can be located proximate to the third region. As such, the second region can separate the first region and the third region in the topology 306.

In some embodiments, the topology 308 of a lattice data structure can include a lattice partitioning with a first region, a second region, and a third region. The first region can include first motors (e.g., a first output region) and the third region can include second motors (e.g. a second output region). Additionally, the second region can include receptors (e.g., an input region) and processors (e.g., a processing region). In some embodiments, respective receptors are separated by respective processors in the second region. The first region can be located proximate to the second region. Additionally, the second region can be located proximate to the third region. As such, the second region can separate the first region and the third region in the topology 308.

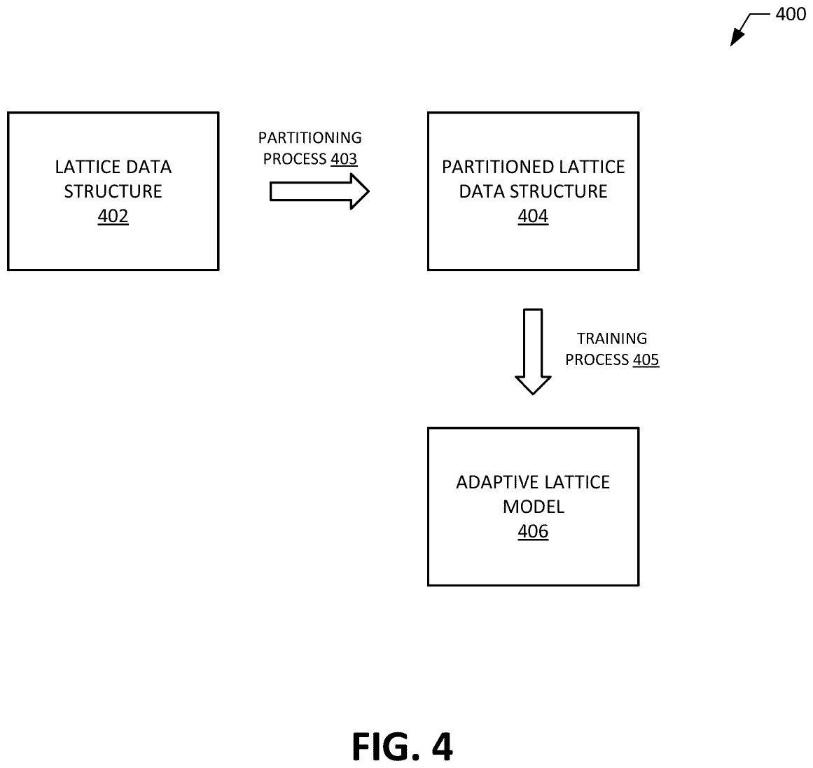

FIG. 4 illustrates a dataflow 400 for training an adaptive lattice model according to one or more embodiments of the present disclosure. The lattice data structure 402 can define a plurality of cells associated with cellular automata. For example, respective cells of the lattice data structure 402 can be associated with an LDM to define a LUT operation for a cellular automaton. Additionally, the LDM can be encoded as a gene. In some embodiments, the lattice data structure 402 can include 1D Cartesian lattice. In some embodiments, the lattice data structure 402 can include a 2D Cartesian lattice.

In some embodiments, the lattice data structure 402 undergoes a partitioning process 403 to provide a partitioned lattice data structure 404. For example, the partitioning process 403 can partition the lattice data structure 402 into an input region (e.g., receptors), an output region (e.g., motors), and a processing region (e.g., processors). In some embodiments, the processing region can be associated with a cost function for the cellular automata. For example, the cost function can utilize a winner-takes-all operation that is applied to states of respective cells of the processing region of the partitioned lattice data structure 404. In some embodiments, the partitioned lattice data structure 404 can include a topology corresponding to the topology 302, the topology 304, the topology 306, or the topology 308.

In some embodiments, the partitioning process 403 may embed an input as an initial condition S(0). Additionally, the lattice data structure 402 can be partitioned into three regions: receptors, motors, and processors so that the state space is not restricted to the input space. In various embodiments, ξ can be an index set of the receptors and o can be another set for the motors such that ξo⊂{1, 2, 3, . . . , N} and |ξ|+|o|≤N. In various embodiments, ξ may be equal to the dimensionality of an input, and K may be larger or equal to the granularity of the input space. For example, if an image is binary, then K may be at least 2. Similarly, the cardinality of o may be larger or equal to the number of classes. If a cell is in neither set, then the cell may correspond to a processor.

The output of the lattice may be determined by the motor states at a time t. Each label may be encoded as a |x|o| one-hot vector. In some embodiments, a temporal window can be applied to the motor states to increase nonlinearity:

$${v}_{i} = {\sum }_{u = 0}^{ω - 1} {s}_{i} ( τ - u ) mod K , ω > 0$$

In some embodiments, a winner-take-all function is applied to each motor state. The index of a motor cell whose state (vi) has the largest value may be considered as a predicted label:

$$y = \{ \begin{matrix}j & if {∀}_{i \neq j ∈ o} {v}_{j} > {v}_{i} \\ 0 & otherwise\end{matrix}$$

In some embodiments, the partitioned lattice data structure 404 can be configured with hyperparameters: A, K, ξ, o, τ, and ω. The hyperparameters can be trainable parameters and/or weights on each neighbor/local connection, including the feedback term w_{i,i}, encoded as non-zero elements in A. Each input can be set to the initial conditions of the receptors. Every cell collectively evolves its internal states through interaction with its neighbors over time. At the t time step, the last w states of the motor cells are read out and applied to the winner-take-all function, determining the output of the lattice.

In some embodiments, a training processing 405 is performed to train an adaptive lattice model 406 based on the partitioned lattice data structure 404. The adaptive lattice model 406 can be a computational model associated with cellular automata. For example, the adaptive lattice model 406 can be a trained cellular automata for a computing task where the trained cellular automata includes multiple processing elements (e.g., cells) over a lattice space. Each processing element (e.g., each cell) can receive its neighbors' state as an input. Additionally, each processing element (e.g., each cell) can evolve its state based on a defined LUT. In some embodiments, the adaptive lattice model 406 can be trained such that local states are mapped over a globally linear lattice associated with the partitioned lattice data structure 404. In some embodiments, the adaptive lattice model 406 can be configured with tuned parameters associated with cortical column functionality that is free from von-Neumann bottlenecks.

In some embodiments, operation of the adaptive lattice model 406 can be defined by a LUT that is tuned according to a training process and/or one or more tunable lookup table operation parameters for the adaptive lattice model 406. For example, the LUT can be specified solely by postsynaptic weights wᵢ, ∈Σ on every ith cell's neighbor:

$${ϕ}_{i} = ( {\sum }_{j ∈ {η}_{i}} {w}_{j} {s}_{j} ( t - 1 ) ) mod K$$

where the adaptive lattice model 406 utilizes a subset of LUTs that hold the additivity over Galois field of order K:

$${∀}_{t > 0} , {ϕ}^{t} \{ {S}_{a} ( 0 ) + {S}_{b} ( 0 ) \} = {ϕ}^{t} \{ {S}_{a} ( 0 ) \} + {ϕ}^{t} \{ {S}_{b} ( 0 ) \}$$

In the vector form, a N×1 state vector S(t) of the lattice at t can be provided as follows:

$$\begin{matrix}S ( t ) = AS ( t - 1 ) mod K \\ = {A}^{t} S ( 0 ) mod K\end{matrix}$$

where A is a N-by-N weighted adjacency matrix, referred to as the characteristic matrix of the adaptive lattice model 406. In some embodiments, A along with K may define a linear cellular automata.

In some embodiments, the adaptive lattice model 406 can be a trained cellular automata for a machine learning task. For example, the adaptive lattice model 406 can be configured to provide one or more predictions, inferences, labels, and/or classifications related to an input dataset using one or more machine learning techniques. In some embodiments, the adaptive lattice model 406 can be a trained cellular automata for an image classification task. For example, the adaptive lattice model 406 can be configured to provide one or more predictions, inferences, labels, and/or classifications related to one or more images. In some embodiments, output of the adaptive lattice model 406 for the machine learning task and/or the image classification task can include a prediction output. The prediction output can be a data construct that describes one or more prediction insights, classifications, and/or inferences provided by the adaptive lattice model 406. In some embodiments, output of the adaptive lattice model 406 for the machine learning task and/or the image classification task can include a binary label. The binary label can be a data construct that classifies data as either a first binary label (e.g., a first class label) or a second binary label (e.g., a second class label) for a particular domain.

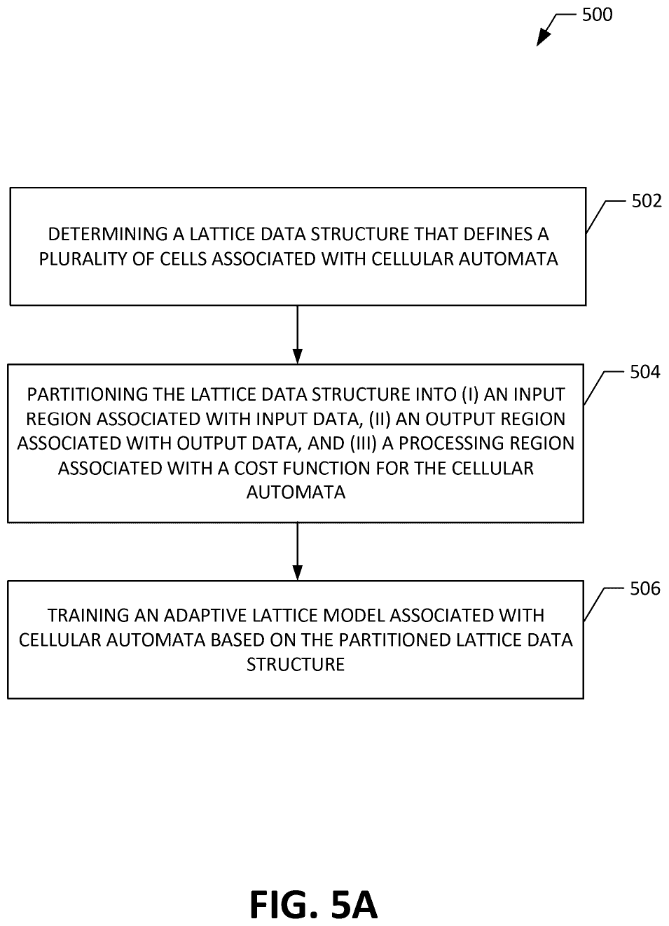

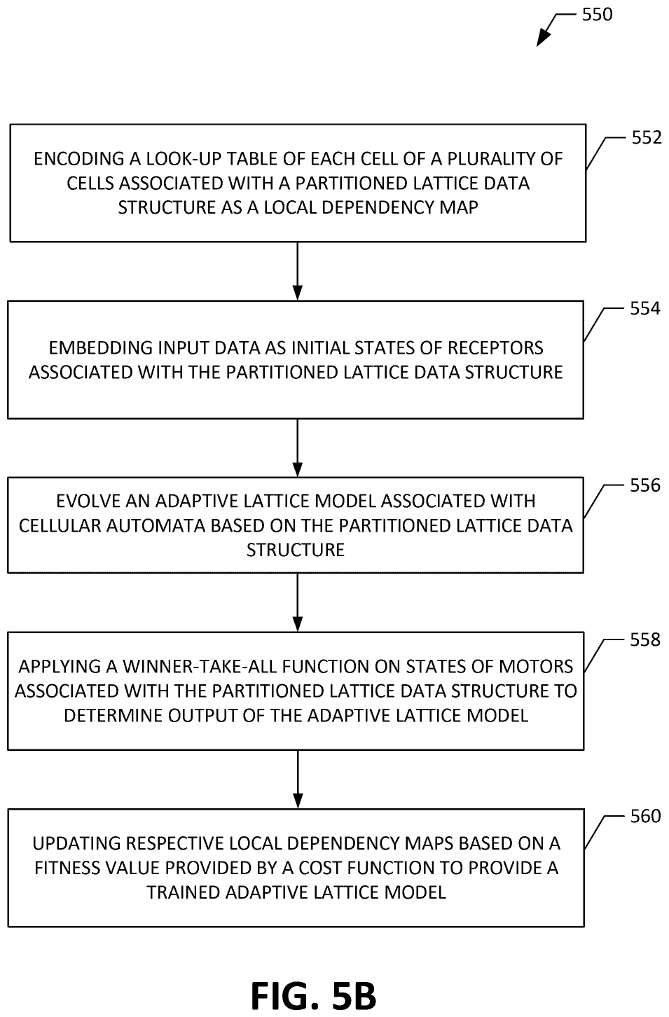

FIG. 5A illustrates a flowchart of a method 500 for providing machine learning associated with cellular automata according to one or more embodiments of the present disclosure. According to the illustrated embodiment, the method 500 includes a step 502 for determining a lattice data structure that defines a plurality of cells associated with cellular automata. Additionally or alternatively, the method 500 includes a step 504 for partitioning the lattice data structure into (i) an input region associated with input data, (ii) an output region associated with output data, and (iii) a processing region associated with a cost function for the cellular automata. In some embodiments, the cost function comprises a winner-takes-all operation that is applied to states of respective cells of the processing region of the partitioned lattice data structure. Additionally or alternatively, the method 500 includes a step 506 for training an adaptive lattice model associated with cellular automata based on the partitioned lattice data structure.

In some embodiments, a local dependency map associated with a look-up table operation for the cellular automata is determined based on the partitioned lattice data structure. In some embodiments, the local dependency map is encoded as a gene.

In some embodiments, the cellular automata is synthesized based on a ranking of chromosome portions of the lattice data structure. In some embodiments, the ranking is based on the cost function.

In some embodiments, training the adaptive lattice model comprises updating the local dependency map based on a fitness value provided by the cost function.

In some embodiments, training the adaptive lattice model comprises applying a winner-take-all function on states of motor cells associated with the partitioned lattice data structure.

In some embodiments, a set of tunable parameters is determined based on the cost function. In some embodiments, the set of tunable parameters is applied to the lattice data structure to determine a next generation of states for the lattice data structure.

In some embodiments, a spatial arrangement of the lattice data structure is configured based on placement of cells and spacing between receptors and processors.

In some embodiments, the performance of the adaptive lattice model is initiated for a machine learning task.

In some embodiments, image classification for a dataset is performed based on the adaptive lattice model.

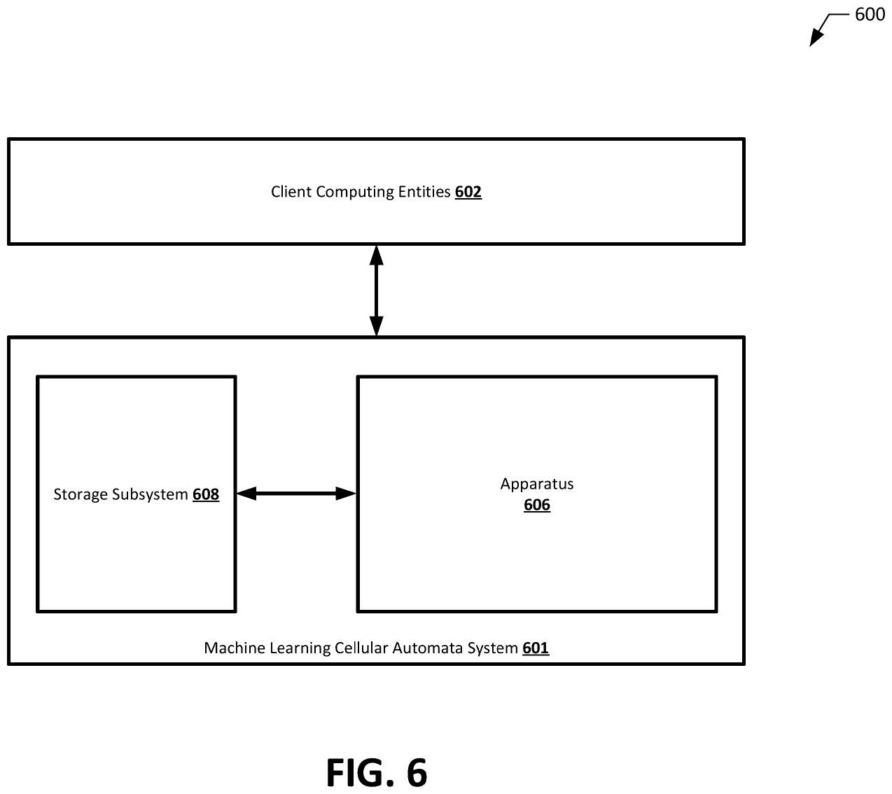

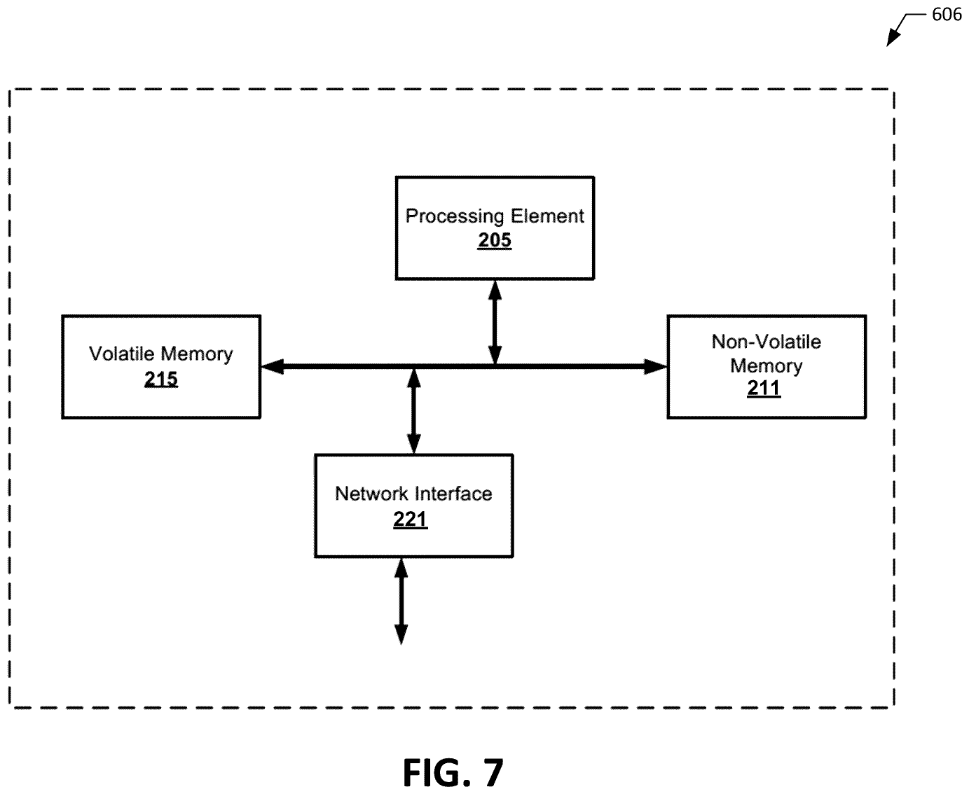

In an example embodiment, an apparatus for performing the method 500 of FIG. 5A above may include a processor configured to perform some or each of the steps 502, 504, and/or 506 described above. The processor may, for example, be configured to perform the steps 502, 504, and/or 506 by performing hardware implemented logical functions, executing stored instructions, or executing algorithms for performing each of the operations. Alternatively, the apparatus may comprise means for performing each of the operations described above. In this regard, according to an example embodiment, examples of means for performing steps 502, 504, and/or 506 may comprise, for example, the processor and/or a device or circuit for executing instructions, executing operations, or executing an algorithm for processing information as described above. In various embodiments, an apparatus for performing the method 500 may correspond to apparatus 606 illustrated in FIG. 6 and/or FIG. 7.

FIG. 5B illustrates a flowchart of a method 550 for training an adaptive lattice model associated with cellular automata according to one or more embodiments of the present disclosure. In some embodiments, the method 550 corresponds to step 506 of the method 500. According to the illustrated embodiment, the method 550 includes a step 552 for encoding a look-up table of each cell of a plurality of cells associated with a partitioned lattice data structure as a local dependency map. Additionally or alternatively, the method 550 includes a step 554 for embedding input data as initial states of receptors (e.g., receptor cells) associated with the partitioned lattice data structure. Additionally or alternatively, the method 550 includes a step 556 for evolve an adaptive lattice model associated with cellular automata based on the partitioned lattice data structure. Additionally or alternatively, the method 550 includes a step 558 for applying a winner-take-all function on states of motors (e.g., motor cells) associated with the partitioned lattice data structure to determine output of the adaptive lattice model. Additionally or alternatively, the method 550 includes a step 560 for updating respective local dependency maps based on a fitness value provided by a cost function to provide a trained adaptive lattice model.

In an example embodiment, an apparatus for performing the method 550 of FIG. 5B above may include a processor configured to perform some or each of the steps 552, 554, 556, 558, and/or 560 described above. The processor may, for example, be configured to perform the steps 552, 554, 556, 558, and/or 560 by performing hardware implemented logical functions, executing stored instructions, or executing algorithms for performing each of the operations. Alternatively, the apparatus may comprise means for performing each of the operations described above. In this regard, according to an example embodiment, examples of means for performing steps 502, 504, and/or 506 may comprise, for example, the processor and/or a device or circuit for executing instructions, executing operations, or executing an algorithm for processing information as described above. In various embodiments, an apparatus for performing the method 550 may correspond to apparatus 606 illustrated in FIG. 6 and/or FIG. 7

### B. Example Technical Implementation of Various Embodiments

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a hardware description language, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD), solid-state card (SSC), solid-state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described with reference to example operations, steps, processes, blocks, and/or the like. Thus, it should be understood that each operation, step, process, block, and/or the like may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

FIG. 6 is a schematic diagram of an example architecture 600 for providing machine learning associated with cellular automata. The architecture 600 includes a machine learning cellular automata system 601 configured to receive predictive data analysis requests from client computing entities 602, process the predictive data analysis requests to train an adaptive lattice model and/or generate predictions using an adaptive lattice model, provide the generated predictions to the client computing entities 602, and automatically perform prediction-based actions based at least in part on the generated predictions.

An example of a prediction-based action that can be performed using the machine learning cellular automata system 601 is a response to a query request for a machine learning task and/or an image classification task. For example, in accordance with various embodiments of the present disclosure, an adaptive lattice model may be trained based on a partitioned lattice data structure to predict responses to queries.

In some embodiments, machine learning cellular automata system 601 may communicate with at least one of the client computing entities 602 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The machine learning cellular automata system 601 may include an apparatus 606 and a storage subsystem 608. The apparatus 606 may be configured to provide machine learning associated with cellular automata using an adaptive lattice model.

The storage subsystem 608 may be configured to store input data (e.g., external memory) used by the apparatus 606 to perform predictive data analysis as well as model definition data used by the apparatus 606 to perform various predictive data analysis tasks. In some embodiments, the storage subsystem 608 can store an adaptive lattice model (e.g., the adaptive lattice model 406) that is trained using a partitioned lattice data structure. The storage subsystem 608 may include one or more storage units, such as multiple distributed storage units that are connected through a computer network. Each storage unit in the storage subsystem 608 may store at least one of one or more data assets and/or one or more data about the computed properties of one or more data assets. Moreover, each storage unit in the storage subsystem 608 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

FIG. 7 provides a schematic of an example apparatus 606 that may be used in accordance with various embodiments of the present disclosure. In particular, the apparatus 606 may be configured to perform various example operations described herein to provide for learning cellular automata. In one or more embodiments, the apparatus 606 may be embodied by one or more portions of the framework 100, a genetic algorithm and/or a training process associated with the system 200, a lattice topology associated with the system 300, and/or the dataflow 400.

In general, the terms computing entity, entity, device, and/or similar words used herein interchangeably may refer to, for example, one or more computers, computing entities, desktop computers, mobile phones, tablets, phablets, notebooks, laptops, distributed systems, items/devices, terminals, servers or server networks, blades, gateways, switches, processing devices, processing entities, set-top boxes, relays, routers, network access points, base stations, or the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

Although illustrated as a single computing entity, those of ordinary skill in the field should appreciate that the apparatus 606 shown in FIG. 7 may be embodied as a plurality of computing entities, tools, and/or the like operating collectively to perform one or more processes, methods, and/or steps. As just one non-limiting example, the apparatus 606 may comprise a plurality of individual data tools, each of which may perform specified tasks and/or processes.

Depending on the embodiment, the apparatus 606 may include one or more network and/or communications interfaces 221 for communicating with various computing entities, such as by communicating data, content, information, and/or similar terms used herein interchangeably that can be transmitted, received, operated on, processed, displayed, stored, and/or the like. Thus, in certain embodiments, the apparatus 606 may be configured to receive data from one or more data sources and/or devices as well as receive data indicative of input, for example, from a device.

The networks used for communicating may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks may have any suitable communication range associated therewith and may include, for example, global networks (e.g., the Internet), MANs, WANs, LANs, or PANs. In addition, the networks may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

Accordingly, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the apparatus 606 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), 5G New Radio (5G NR), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The apparatus 606 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

In addition, in various embodiments, the apparatus 606 includes or is in communication with one or more processing elements 205 (also referred to as processors, processing circuitry, and/or similar terms used herein interchangeably) that communicate with other elements within the apparatus 606 via a bus, for example, or network connection. As will be understood, the processing element 205 may be embodied in several different ways. For example, the processing element 205 may be embodied as one or more complex programmable logic devices (CPLDs), microprocessors, multi-core processors, coprocessing entities, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, ASICs, FPGAs, programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware, computer program products, or a combination thereof, the processing element 205 may be capable of performing steps or operations according to embodiments of the present disclosure when configured accordingly.

In various embodiments, the apparatus 606 may include or be in communication with non-volatile media (also referred to as non-volatile storage, memory, memory storage, memory circuitry and/or similar terms used herein interchangeably). For instance, the non-volatile storage or memory may include one or more non-volatile storage or non-volatile memory media 211 such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or non-volatile memory media 211 may store files, databases, database instances, database management system entities, images, data, applications, programs, program modules, scripts, source code, object code, byte code, compiled code, interpreted code, machine code, executable instructions, and/or the like. The term database, database instance, database management system entity, and/or similar terms used herein interchangeably and in a general sense refer to a structured or unstructured collection of information/data that is stored in a computer-readable storage medium.

In particular embodiments, the non-volatile memory media 211 may also be embodied as a data storage device or devices, as a separate database server or servers, or as a combination of data storage devices and separate database servers. Further, in some embodiments, the non-volatile memory media 211 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. As already discussed, various embodiments contemplated herein use data storage in which some or all the information/data required for various embodiments of the disclosure may be stored.

In various embodiments, the apparatus 606 may further include or be in communication with volatile media (also referred to as volatile storage, memory, memory storage, memory circuitry and/or similar terms used herein interchangeably). For instance, the volatile storage or memory may also include one or more volatile storage or volatile memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like.

As will be recognized, the volatile storage or volatile memory media 215 may be used to store at least portions of the databases, database instances, database management system entities, data, images, applications, programs, program modules, scripts, source code, object code, byte code, compiled code, interpreted code, machine code, executable instructions, and/or the like being executed by, for example, the processing element 205. Thus, the databases, database instances, database management system entities, data, images, applications, programs, program modules, scripts, source code, object code, byte code, compiled code, interpreted code, machine code, executable instructions, and/or the like may be used to control certain aspects of the operation of the apparatus 606 with the assistance of the processing element 205 and operating system.

As will be appreciated, one or more of the computing entity's components may be located remotely from the other computing entity components, such as in a distributed system. Furthermore, one or more of the components may be aggregated, and additional components performing functions described herein may be included in the apparatus 606. Thus, the apparatus 606 can be adapted to accommodate a variety of needs and circumstances.

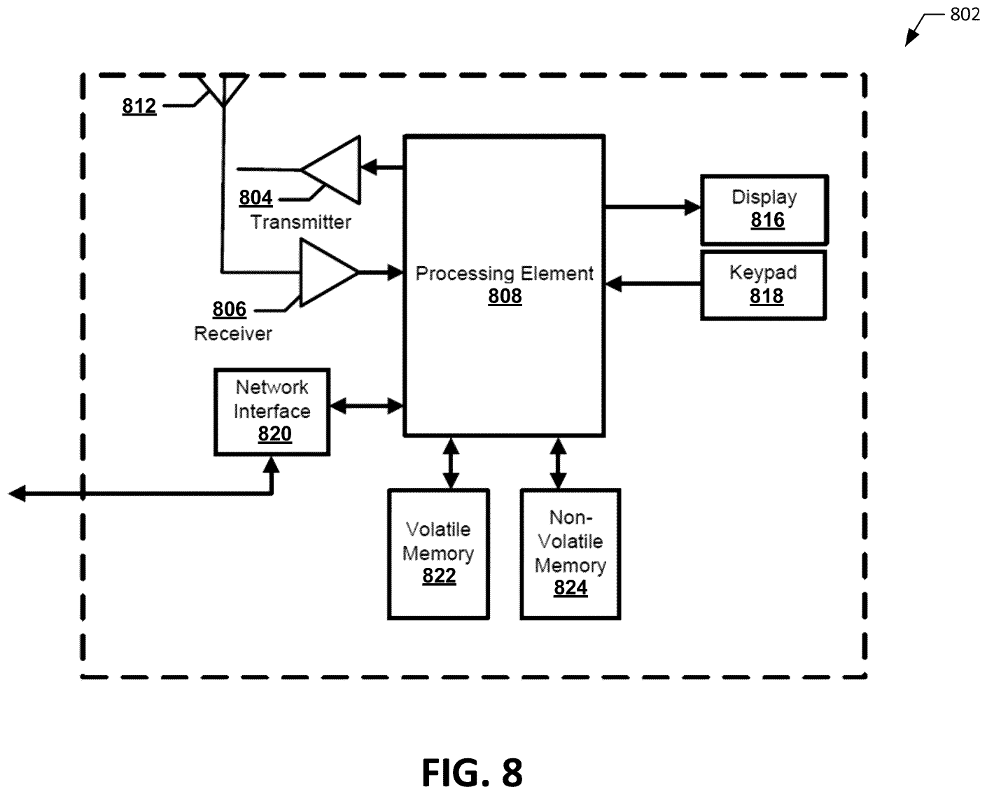

FIG. 8 provides an illustrative schematic representative of a client computing entity 602 that can be used in conjunction with embodiments of the present disclosure. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 602 can be operated by various parties. As shown in FIG. 8, the client computing entity 602 can include an antenna 812, a transmitter 804 (e.g., radio), a receiver 806 (e.g., radio), and a processing element 808 (e.g., CPLDs, microprocessors, multi-core processors, coprocessing entities, ASIPs, microcontrollers, and/or controllers) that provides signals to and receives signals from the transmitter 804 and receiver 806, correspondingly.

The signals provided to and received from the transmitter 804 and the receiver 806, correspondingly, may include signaling information/data in accordance with air interface standards of applicable wireless systems. In this regard, the client computing entity 602 may be capable of operating with one or more air interface standards, communication protocols, modulation types, and access types. More particularly, the client computing entity 602 may operate in accordance with any of a number of wireless communication standards and protocols, such as those described above with regard to the apparatus 606. In a particular embodiment, the client computing entity 602 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 602 may operate in accordance with multiple wired communication standards and protocols, such as those described above with regard to the apparatus 606 via a network interface 820.

Via these communication standards and protocols, the client computing entity 602 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 602 can also download changes, add-ons, and updates, for instance, to its firmware, software (e.g., including executable instructions, applications, program modules), and operating system.

According to one embodiment, the client computing entity 602 may include location determining aspects, devices, modules, functionalities, and/or similar words used herein interchangeably. For example, the client computing entity 602 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 302 position in connection with a variety of other systems, including cellular towers, Wi-Fi access points, and/or the like. Similarly, the client computing entity 602 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 602 may also comprise a user interface (that can include a display 816 coupled to a processing element 808) and/or a user input interface (coupled to a processing element 808). For example, the user interface may be a user application, browser, user interface, and/or similar words used herein interchangeably executing on and/or accessible via the client computing entity 602 to interact with and/or cause display of information/data from the apparatus 606, as described herein. The user input interface can comprise any of a number of devices or interfaces allowing the client computing entity 602 to receive data, such as a keypad 818 (hard or soft), a touch display, voice/speech or motion interfaces, or other input device. In embodiments including a keypad 818, the keypad 818 can include (or cause display of) the conventional numeric (0-9) and related keys (#, *), and other keys used for operating the client computing entity 602 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 602 can also include volatile memory 822 and/or non-volatile memory 824, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 602. As indicated, this may include a user application that is resident on the entity or accessible through a browser or other user interface for communicating with the apparatus 606 and/or various other computing entities.

In another embodiment, the client computing entity 602 may include one or more components or functionality that are the same or similar to those of the apparatus 606, as described in greater detail above. As will be recognized, these architectures and descriptions are provided for exemplary purposes only and are not limiting to the various embodiments.

In various embodiments, the client computing entity 602 may be embodied as an artificial intelligence (AI) computing entity. Accordingly, the client computing entity 602 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### D. Example Topology for a Partitioned Lattice Data Structure

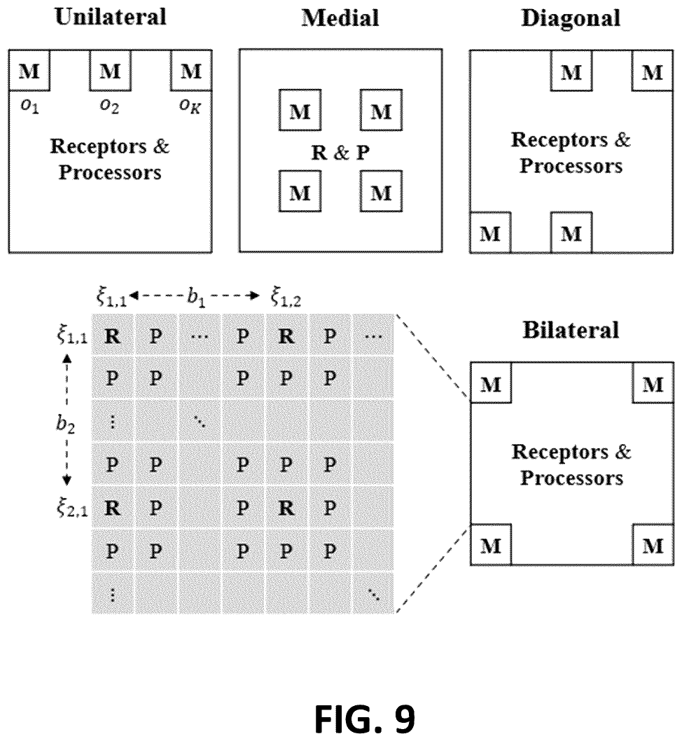

FIG. 9 illustrates example topologies for a partitioned lattice data structure according to one or more embodiments of the present disclosure. For example, FIG. 9 can illustrate an example of 2D lattice partitioning using a topology based on the placement of motor cells for a 2D lattice associated with a partitioned lattice data structure. In some embodiments, a partitioned lattice data structure associated with a topology illustrated in FIG. 9 can be utilized to train the adaptive lattice model 406. For example, a topology for a partitioned lattice data structure utilized to train the adaptive lattice model 406 can correspond to a unilateral topology, a medial topology, a diagonal topology, or a bilateral topology. In some embodiments, a topology can be associated with a von-Neumann neighborhood or a Moore neighborhood. In some embodiments, Monte Carlo simulation can be utilized to estimate the density of solutions given different topologies of the lattices. For each Boolean operator, 106 chromosomes are generated by randomly drawing nucleotides from a uniform distribution of 0, 1, 2. Then, the fitness is computed on every chromosome. The lattice settings are N=16, K=8, τ=50, and ω=1. The hyperparameters, r, ξ, and o, are determined by a lattice topology. A number of unique solutions for different topologies is illustrated below:

| | | | | 2D | | | | | |
|---|---|---|---|---|---|---|---|---|---|
| | 1D (r = 2) | | | von-Neumann (r = 1) | | von-Neumann (r = 2) | | Moore (r = 1) | |
| | Unilateral | Medial | Bilateral | Unilateral | Bilateral | Unilateral | Bilateral | Unilateral | Bilateral |
| NAND | 2.150 | 3.473 | 4.585 | 1.863 | 2.073 | 23.465 | 23.920 | 16.829 | 16.701 |
| AND | 2.258 | 3.476 | 4.495 | 1.877 | 2.130 | 23.577 | 23.842 | 16.413 | 16.798 |
| NOR | 2.201 | 3.566 | 4.553 | 1.830 | 2.125 | 23.413 | 23.985 | 16.623 | 16.619 |
| OR | 2.093 | 3.649 | 4.551 | 1.872 | 2.084 | 23.691 | 23.841 | 16.315 | 16.693 |
| XNOR | 5.472 | 3.228 | 7.988 | 4.305 | 4.553 | 27.868 | 28.047 | 23.205 | 22.636 |
| XOR | 5.535 | 3.286 | 8.023 | 4.433 | 4.517 | 27.652 | 28.056 | 23.023 | 22.541 |
| Average | 3.285 | 3.446 | 5.699 | 2.696 | 2.914 | 24.944 | 25.282 | 18.735 | 18.665 |

×10⁻ [symbol] [symbol]
indicates data missing or illegible when filed

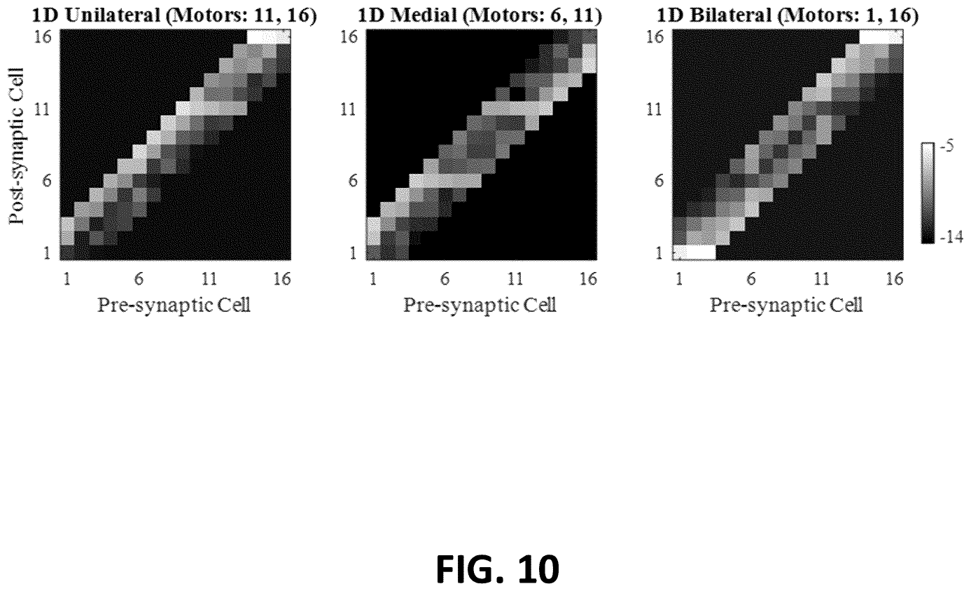

Additionally, it is to be appreciated that the parameter size of different neighborhoods can grow on a 2D lattice while constantly on a 1D lattice. For image classification, logarithm of the average mutual information between each cell and the fitness value for the NAND operator is illustrated in FIG. 10. As illustrated in FIG. 10, the motor's post-synaptic weights (e.g., input towards the cell, a horizontal line) have the highest information content. In contrast, the receptors have a higher information content on their pre-synaptic weights (e.g., output from the cell, a vertical line). The receptor locations can be 1 and 6 for the unilateral topology (a), 1 and 16 for the medial topology (b), and 6 and 11 for the bilateral topology (c). In an example, the receptors can be located from 5 and 10 cells away from each motor. As such, by utilizing a partitioned lattice data structure associated with a topology illustrated in FIG. 9 (e.g., a 2D lattice), a greater number of solutions per number of neighbors can be provided. Moreover, a partitioned lattice data structure associated with a topology illustrated in FIG. 9 (e.g., a 2D lattice) can increase the degree of freedom in fining a path to an input-output map through space-time. In various embodiments, a partitioned lattice data structure associated with a topology illustrated in FIG. 9 (e.g., a 2D lattice) can preserve spatial structures of input. Additionally, a partitioned lattice data structure associated with a topology illustrated in FIG. 9 (e.g., a 2D lattice) can provide a higher degree of freedom per weight/parameter.

### E. Conclusion

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for providing machine learning associated with cellular automata, the method comprising:
determining a lattice data structure that defines a plurality of cells associated with cellular automata;
partitioning the lattice data structure into (i) an input region associated with input data, (ii) an output region associated with output data, and (iii) a processing region associated with a cost function for the cellular automata; and
training an adaptive lattice model associated with cellular automata based on the partitioned lattice data structure.

2. The method of claim 1, wherein the cost function comprises a winner-takes-all operation that is applied to states of respective cells of the processing region.

3. The method of claim 1, further comprising:
determining a local dependency map associated with a look-up table operation for the cellular automata based on the partitioned lattice data structure.

4. The method of claim 3, further comprising:
encoding the local dependency map as a gene.

5. The method of claim 3, wherein training the adaptive lattice model comprises updating the local dependency map based on a fitness value provided by the cost function.

6. The method of claim 3, wherein training the adaptive lattice model comprises applying a winner-take-all function on states of motor cells associated with the partitioned lattice data structure.

7. The method of claim 1, further comprising:
determining a set of tunable parameters based on the cost function; and
applying the set of tunable parameters to the lattice data structure to determine a next generation of states for the lattice data structure.

8. The method of claim 1, further comprising:
configuring a spatial arrangement of the lattice data structure based on placement of cells and spacing between receptors and processors.

9. The method of claim 1, further comprising:
initiating the performance of the adaptive lattice model for a machine learning task.

10. The method of claim 1, further comprising:
performing image classification for a dataset based on the adaptive lattice model.

11. An apparatus comprising at least one processor and at least one memory including program code, the at least one memory and the program code configured to, with the at least one processor, cause the apparatus to at least:
determine a lattice data structure that defines a plurality of cells associated with cellular automata;
partition the lattice data structure into (i) an input region associated with input data, (ii) an output region associated with output data, and (iii) a processing region associated with a cost function for the cellular automata; and
train an adaptive lattice model associated with cellular automata based on the partitioned lattice data structure.

12. The apparatus of claim 11, wherein the cost function comprises a winner-takes-all operation that is applied to states of respective cells of the processing region.

13. The apparatus of claim 11, wherein the at least one memory and the program code are configured to, with the at least one processor, further cause the apparatus to at least:
determine a local dependency map associated with a look-up table operation for the cellular automata based on the partitioned lattice data structure.

14. The apparatus of claim 13, wherein the at least one memory and the program code are configured to, with the at least one processor, further cause the apparatus to at least:
encode the local dependency map as a gene.

15. The apparatus of claim 13, wherein the at least one memory and the program code are configured to, with the at least one processor, further cause the apparatus to at least:
train the adaptive lattice model comprises updating the local dependency map based on a fitness value provided by the cost function.

16. The apparatus of claim 13, wherein the at least one memory and the program code are configured to, with the at least one processor, further cause the apparatus to at least:
apply a winner-take-all function on states of motor cells associated with the partitioned lattice data structure.

17. The apparatus of claim 11, wherein the at least one memory and the program code are configured to, with the at least one processor, further cause the apparatus to at least:
determine a set of tunable parameters based on the cost function; and
apply the set of tunable parameters to the lattice data structure to determine a next generation of states for the lattice data structure.

18. The apparatus of claim 11, wherein the at least one memory and the program code are configured to, with the at least one processor, further cause the apparatus to at least:
configure a spatial arrangement of the lattice data structure based on placement of cells and spacing between receptors and processors.

19. The apparatus of claim 18, wherein the at least one memory and the program code are configured to, with the at least one processor, further cause the apparatus to at least:
initiate the performance of the adaptive lattice model for a machine learning task.

20. A non-transitory computer storage medium comprising instructions, the instructions being configured to cause one or more processors to at least perform operations configured to:
determine a lattice data structure that defines a plurality of cells associated with cellular automata;
partition the lattice data structure into (i) an input region associated with input data, (ii) an output region associated with output data, and (iii) a processing region associated with a cost function for the cellular automata; and
train an adaptive lattice model associated with cellular automata based on the partitioned lattice data structure.
